(19) United States
(12) Patent Application Publication
(10) Pub. No.: US 2026/0122057 A1
(43) Pub. Date: Apr. 30, 2026

(54) KEY MANAGEMENT FOR MEDICAL DEVICES

(71) Applicant: Bardy Diagnostics, Inc., Bellevue, WA (US)

(72) Inventors: Justin Hynes-Bruell, Bellevue, WA (US); Ezra M. Dreisbach, Bellevue, WA (US)

(21) Appl. No.: 19/369,712

(22) Filed: Oct. 27, 2025

Related U.S. Application Data
(60) Provisional application No. 63/711,955, filed on Oct. 25, 2024.

(51) Int. Cl. H04L 9/40; A61B 5/00; G16H 40/67

(52) U.S. Cl. CPC H04L 63/083; A61B 5/0006; G16H 40/67

(57) ABSTRACT

A system includes a key service, that is configured to authenticate requests from an external manufacturing system (“EMS”), receive a request for generating a keyset, generate the keyset, send the keyset to the EMS, and send the keyset to a persistent cloud storage system, wherein an external cloud system accesses the keyset for use in secure communication with a hardware device.

### PRIORITY CLAIM AND CROSS-REFERENCE TO RELATED APPLICATIONS

This application claims priority to, and the benefit of, U.S. Provisional Patent Application No. 63/711,955, filed Oct. 25, 2024, titled “KEY MANAGEMENT FOR MEDICAL DEVICES,” the entire contents of which are incorporated by reference herein in their entirety and relied upon.

### FIELD

This application relates in general to key management for medical devices, such as electrocardiogramonitoring devices or more particularly Mobile Cardiac Telemetry (“MCT”) devices.

### BACKGROUND

Medical devices, such as MCT systems may transmit patient data to other networks, systems and databases. The other networks, systems and databases may be part of a cloud-based system or cloud-based computing platform. MCT devices are configured to function as an ECG with the additional benefit of transmitting ECG data in close to real-time for analysis by networked systems. This connectivity allows for faster feedback to patients and/or health care providers with respect to, for example, arrhythmias patients may have experienced. By comparison, most traditional ECG systems simply record ECG data for later analysis, failing to provide more dynamic feedback and leading to a longer delay in care. An electrocardiogram (“ECG”) measures and records such electrical potentials to visually depict the electrical activity of the heart over time. Conventionally, a standardized set format 12-lead configuration is used by an ECG machine to record cardiac electrical signals from well-established traditional chest locations. Electrodes at the end of each lead are placed on the skin over the anterior thoracic region of the patient's body to the lower right and to the lower left of the sternum, on the left anterior chest, and on the limbs. Sensed cardiac electrical activity is represented by PQRSTU waveforms that can be interpreted post-ECG recordation to derive heart rate and physiology. The P-wave represents atrial electrical activity. The QRSTU components represent ventricular electrical activity.

An ECG is a tool used by physicians to diagnose heart problems and other potential health concerns. An ECG is a snapshot of heart function, typically recorded over 12 seconds, that can help diagnose rate and regularity of heartbeats, effect of drugs or cardiac devices, including pacemakers and implantable cardioverter-defibrillators (“ICDs”), and whether a patient has heart disease.

Medical devices, such as MCT devices operating as a longer term connected ECG monitors, may have sensitive patient data that needs to be handled with care. In the case of an MCT device, this data is communicated over a network to a cloud-computing environment. Moreover, with the MCT device, it is critical that the device is secure, such that recorded data saved on the device in either a permanent or buffered state is secure and maintains its integrity. Further, with the MCT device, it is important to ensure that the software running on the device is protected and authentic. Therefore, a need remains for a secure method to generate and manage secure assets, such as keys, certificates, IDs, and serial numbers, for communication between a medical device hardware and an associated cloud computing environment. For convenience, as used herein, the term key or keys is generally intended to describe these secure assets of any one or more of keys, certificates, IDs, Firmware and serial numbers.

### SUMMARY

One embodiment provides a key management system. The system includes a key service, and a key store. The key service is configured to authenticate RESTful requests for keys from an external manufacturing system (“EMS”). In an embodiment, this authentication is performed via certificates/passwords. In an alternative embodiment, this authentication is performed via tokens. The key service generates key(s) upon each GET request from an EMS. The keys generated are simultaneously stored in a key store database, where they are available to downstream cloud Software systems. The keys provided to the EMS may be subsequently programmed (e.g., after generation and storage) into MCT hardware devices; these keys can therefore be used for end-to-end encryption between hardware and the before mentioned cloud software systems.

In an example, which may be used in combination with any one or more of the other example embodiments described herein, provides a method which includes receiving, by a key service.

Still other embodiments will become readily apparent to those skilled in the art from the following detailed description, wherein are described embodiments by way of illustrating the best mode contemplated. As will be realized, other and different embodiments are possible and the embodiments' several details are capable of modifications in various obvious respects, all without departing from their spirit and the scope. Accordingly, the drawings and detailed description are to be regarded as illustrative in nature and not as restrictive.

In light of the disclosure set forth herein, and without limiting the disclosure in any way, in a first aspect of the present disclosure, which may be combined with any other aspect, or portion thereof, a system includes a key service, that is configured to authenticate requests from an external manufacturing system (“EMS”), receive a request for generating a keyset, generate the keyset, send the keyset to the EMS, and send the keyset to a persistent cloud storage system, wherein an external cloud system accesses the keyset for use in secure communication with a hardware device.

In a second aspect of the present disclosure, which may be combined with any other aspect, or portion thereof, the keyset includes a plurality of keys.

In a third aspect of the present disclosure, which may be combined with any other aspect, or portion thereof, the plurality of keys includes at least seven keys.

In a fourth aspect of the present disclosure, which may be combined with any other aspect, or portion thereof, the system further includes a key storage memory configured to store the keyset.

In a fifth aspect of the present disclosure, which may be combined with any other aspect, or portion thereof, the key storage memory is configured to store each key of the keyset together as a unit in key storage memory.

In a sixth aspect of the present disclosure, which may be combined with any other aspect, or portion thereof, the key storage memory is persistent memory.

In a seventh aspect of the present disclosure, which may be combined with any other aspect, or portion thereof, the keyset includes at least one of a device identifier, a hardware identifier, an encryption key, an image authentication key, an image signature, a serial key, a root key, and an image encryption key.

In an eighth aspect of the present disclosure, which may be combined with any other aspect, or portion thereof, the key service is configured to send the generated keyset as an array, wherein the array is a JSON formatted array.

In a ninth aspect of the present disclosure, which may be combined with any other aspect, or portion thereof, a method includes authenticating, by a key service, requests from an external manufacturing system (“EMS”), receiving, by the key service, a request for generating a keyset, generating, by the key service, the keyset, sending the keyset to the EMS, and sending the keyset to a persistent cloud storage system, wherein an external cloud system accesses the keyset for use in secure communication with a hardware device.

In a tenth aspect of the present disclosure, which may be combined with any other aspect, or portion thereof, the keyset includes a plurality of keys.

In an eleventh aspect of the present disclosure, which may be combined with any other aspect, or portion thereof, the plurality of keys includes at one of a device identifier, a hardware identifier, an encryption key, an image authentication key, an image signature, a serial key, a root key, and an image encryption key.

In a twelfth aspect of the present disclosure, which may be combined with any other aspect, or portion thereof, each key of the keyset is stored together as a unit in key storage memory.

In a thirteenth aspect of the present disclosure, which may be combined with any other aspect, or portion thereof, the key storage memory is persistent memory.

In a fourteenth aspect of the present disclosure, which may be combined with any other aspect, or portion thereof, the array is a JSON formatted array.

In a fifteenth aspect of the present disclosure, which may be combined with any other aspect, or portion thereof, a respective key of the keyset has a source, and wherein the source is one of a configured source and a random source.

In a sixteenth aspect of the present disclosure, which may be combined with any other aspect, or portion thereof, the configured source is configured to provide preloaded keys and manually set keys, wherein the random source includes a cryptographically secure random number generator.

In a seventeenth aspect of the present disclosure, which may be combined with any other aspect, or portion thereof, a non-transitory machine readable medium storing code, which when executed by a processor causes a key service to authenticate requests from an external manufacturing system (“EMS”), receive a request for generating a keyset, generate the keyset, send the keyset to the EMS, and send the keyset to a persistent cloud storage system, wherein an external cloud system accesses the keyset for use in secure communication with a hardware device.

In an eighteenth aspect of the present disclosure, which may be combined with any other aspect, or portion thereof, the keyset includes a plurality of keys.

In a nineteenth aspect of the present disclosure, which may be combined with any other aspect, or portion thereof, the plurality of keys includes at one of a device identifier, a hardware identifier, an encryption key, an image authentication key, an image signature, a serial key, a root key, and an image encryption key.

In a twentieth aspect of the present disclosure, which may be combined with any other aspect, or portion thereof, each key of the keyset is stored together as a unit in key storage memory, and wherein the key storage memory is persistent memory.

Still other embodiments will become readily apparent to those skilled in the art from the following detailed description, wherein are described embodiments by way of illustrating the best mode contemplated. As will be realized, other and different embodiments are possible and the embodiments' several details are capable of modifications in various obvious respects, all without departing from their spirit and the scope. Accordingly, the drawings and detailed description are to be regarded as illustrative in nature and not as restrictive.

### BRIEF DESCRIPTION OF THE DRAWINGS

FIG. 1 is a block diagram of a key management system, according to an example of the present disclosure.

FIG. 2 is a block diagram of a key management system, according to an example of the present disclosure.

FIG. 3 is a flowchart of an example mutual authentication process, according to an example of the present disclosure.

FIG. 4A is a flow diagram of an example process for a successful mutual authentication between an external manufacturing system and a key service using a password-token authority and table storage according to an example embodiment of the present disclosure.

FIG. 4B is a flow diagram of an example process for a failed mutual authentication between an external manufacturing system and a key service using a password-token authority and table storage according to an example embodiment of the present disclosure.

FIG. 5 is a flow diagram of an example process for keyset generation and management, according to an example of the present disclosure.

FIG. 6 is an example keyset array, according to an example of the present disclosure.

### DETAILED DESCRIPTION

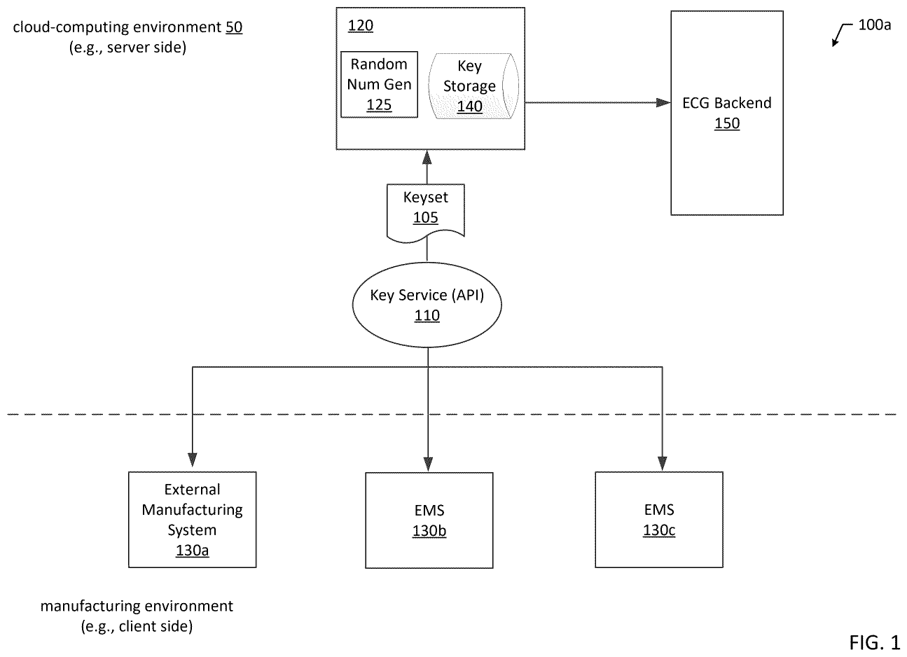

As illustrated by the Figures herein, starting with FIG. 1, techniques are disclosed for providing key generation, key management, and authentication using a key service and related key store for medical devices (e.g., hardware) and applications (e.g., web applications, ECG processing systems). In an example embodiment, a system includes a key service (API) 110, and a key store 140. The key service 110 is configured to authenticate requests from an external manufacturing system (“EMS”) 130a and to provide keysets to the EMS 130a. In an embodiment, keysets sent from the key service 110 may include common data that is to be install/programmed/configured into hardware devices by the EMS 130a, 130b, 130c, including, but not limited to, firmware images, keys, certificates and other secure assets.

Simultaneously to transmitting keysets to an EMS 130a, some or all of the keys are stored in the key store 140 for use by other cloud systems (e.g., ECG backend 150) for communication and management of hardware devices. In an embodiment, key store 140 is a persistent cloud storage system.

When said keysets are programmed into hardware, and that hardware wishes to communicate with cloud systems such as the ECG backend 150, it may use these keys to send end-to-end encrypted and signed data to enable the secure transfer of information such as patient ECG data.

Keys provided to the EMS 130a are installed in medical hardware such as the MCT. With these keys installed, the medical device can now, for example, send a networked message/packet to the cloud environment 50. This packet may include the Device ID in clear text, then an encrypted and signed data. The cloud system would then look up the keys to decrypt and authenticate the message, using the key store with the associated Device ID as a look up in the table. With this information the cloud system would authenticate, decrypt, and process the data packet. Having the system setup this way, with symmetric keys unique to each device, enables secure end-to-end communication between the medical device and cloud systems.

In an example, the key service may be limited to a predetermined maximum number of keysets generated per time interval, such as five thousand keysets per day, per client. It should be appreciated that other limits on keyset generation may be applied. Each client that calls the API to the key service requires a unique client token.

A key may be 48, 96, or 128 bits and may be encoded in JSON data as hex strings. For example, “key1” may be a 48-bit key “a5a5a5a5a5a5” and “key2” may be a 128-bit key “a5a5a5a5a5a5a5a5a5a5a5a5a5a5a5a5.” The keys or keysets may be sourced from various types of sources including a configured source(s) and a random source(s). Keys or keysets from a configured source are the same in every generated keyset. For example, these keys may be pre-loaded from a key service (e.g., table storage) and may be manually set by an administrator. Keys from random sources are randomly generated, such as from a cryptographically secure random number generator. Randomly generated keys may be generated using a random number generator (“RNG”), a pseudorandom number generator (“PRNG”), or a combination thereof. In alternative embodiments, a counter may be implemented for key generation.

Regarding providing the keys in a cryptographically secure manner, the key service or key generator may use one or more encryption standards, such as the Data Encryption Standard (“DES”), the Advanced Encryption Standard (“AES”), the RSA cryptosystem, or the like. It should be appreciated that other data standards, encryption standards and/or public-key systems may be used. The key service may be configured to provide features and services while maintaining data security and data integrity.

More specifically, referring to FIG. 1, the system 100a includes a key service 110 and other services or components 120 (e.g., a database or memory), such as a random number generator 125 and key storage 140. The key service 110 and other services or components 120 may communicate with a backend software component (“ECGBE”) of a medical device, such as an ECG or MCT device. Each of the above components and services may run in a cloud-computing environment (“CCE”) 50. In an example, the key service 110 may also communicate with various external manufacturing systems (“EMS”) 130. In the illustrated example, there are three EMSs 130a, 130b, and 130c, but it should be appreciated that the system 100 may communicate with more or less EMS(s) 130. The random number generator 125 and key storage 140 are configured to persistently store the key counts and keysets. Preferentially, random number generator 125 is stateless. In an alternative embodiment, random number generator 125 is configured to generate keys sequentially (e.g., via a counter or other related means).

The key service 110 may be configured to generate one or more keyset(s) 105, which may be communicated to other devices and systems illustrated in FIG. 1, save the keyset in key storage 140, and send the keyset 105 to authenticated devices and parties (e.g., EMS 130). In an example, the key service or key service 110 is a web service that is hosted in a cloud-computing environment 50 and is configured to generate unique keysets, store keysets into the random number generator 125 and/or key storage 140, and provide an API for each EMS 130 such that each EMS 130 can request keysets. Typically, an EMS 130 calls the API to get a collection of keysets from the key service 110.

As noted above, the ECGB 150 may be a software component within a medical device or medical device system. The ECGB 150 may access a keyset stored in the key storage 140 to obtain a valid device serial number. It should be appreciated that each client (e.g., each EMS 130) that calls the API to the key service 110 may require a unique client token.

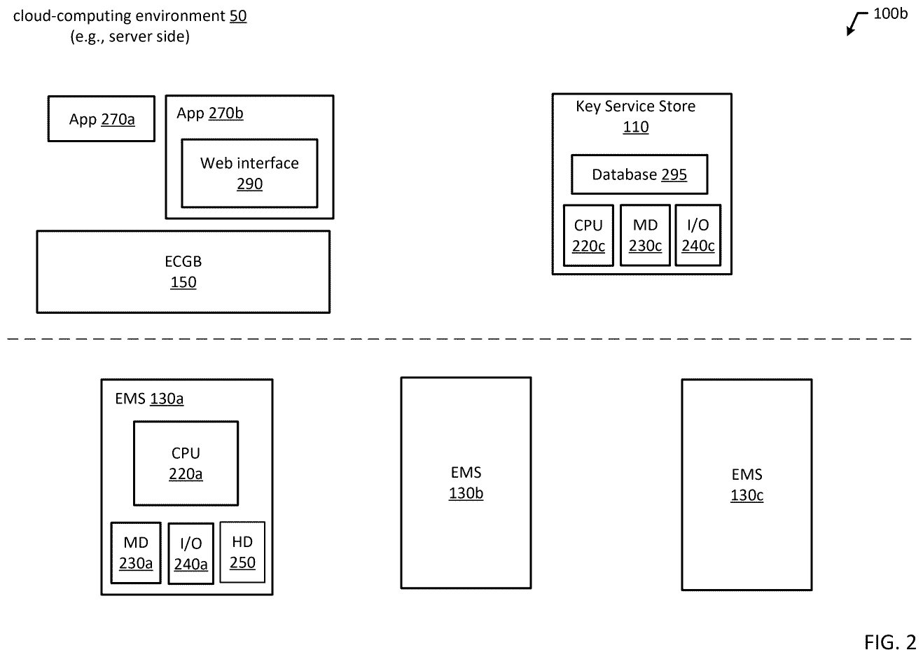

FIG. 2 illustrates another example system 100b in accordance with one or more aspects of the present disclosure. The system 100b may include one or more EMS(s) 130 that are configured to communicate with various components of the CCE 50. Each EMS 130 may be a computer, appliance, other network device, or the like that is capable of communicating with other machines over a network. In an example, an EMS 130 (e.g., EMS 130a) may include one or more physical processors (e.g., CPU 220a), memory devices 230 (e.g., MD 230a), input/output devices 240 (e.g., I/O 240a), and hardware devices 250. A hardware device 250 may include a network device (e.g., a network interface controller (NIC), a network adapter, or any other component that connects a computer to a computer network), a peripheral component interconnect (PCI) device, storage devices, sound or video adaptors, photo/video cameras, printer devices, keyboards, displays, etc.

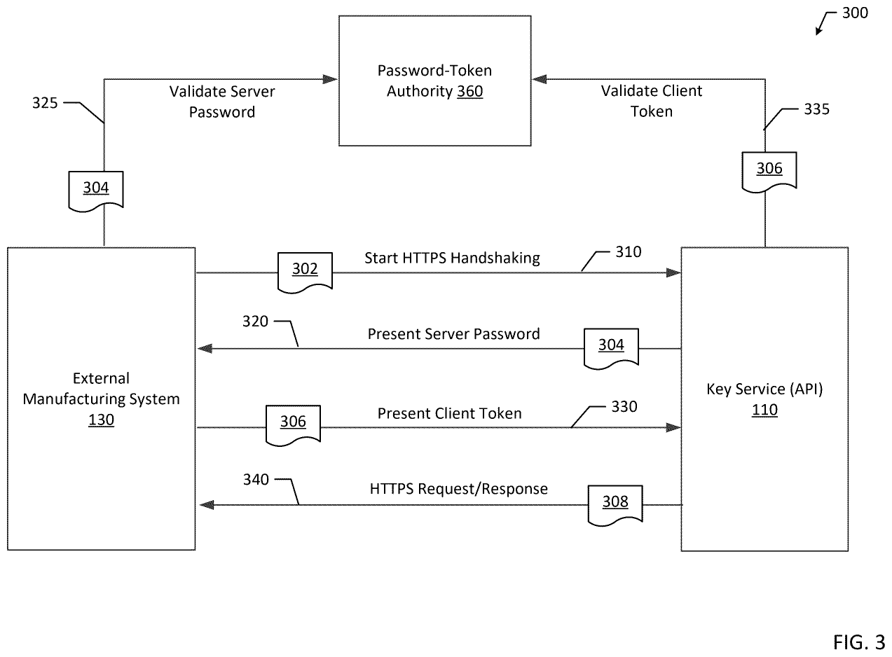

In an embodiment, the system 100b may also include a password-token authority (e.g., as illustrated in FIG. 3). For example, this authority may in turn include one or more physical processors communicatively coupled to memory devices and input/output devices. The system 100b may include a key service 110, which may be generally referred to as a key service 110.

In an example, the key service or key service 110 may include a database 295, one or more physical processors (e.g., CPU 220c) communicatively coupled to a memory device (e.g., MD 230c), and input/output devices (e.g., I/O 240c). The key service 110, which may serve as a password-token authority, may be responsible for managing authentication and authorization between and on behalf of the EMS(s) 130, key service 110, and any applications (e.g., Applications 270a-b) or interfaces (e.g., ECGB 150) of medical devices, such as an MCT device. The application 170C may also include a web interface 290.

As used herein, physical processor or processor 230 refers to a device capable of executing instructions encoding arithmetic, logical, and/or I/O operations. A processor may follow Von Neumann architectural model and may include an arithmetic logic unit (“ALU”), a control unit, and a plurality of registers. A processor may be a single core processor which is typically capable of executing one instruction at a time (or process a single pipeline of instructions), or a multi-core processor which may simultaneously execute multiple instructions. In another aspect, a processor may be implemented as a single integrated circuit, two or more integrated circuits, or may be a component of a multi-chip module (e.g., in which individual microprocessor dies are included in a single integrated circuit package and hence share a single socket). A processor may also be referred to as a central processing unit (“CPU”).

As discussed herein, a memory device 230 refers to a volatile or non-volatile memory device, such as random-access memory (“RAM”), read-only memory (“ROM”), electrically-erasable-programmable read-only memory (“EEPROM”), or any other device capable of storing data. As discussed herein, I/O device 240 may refer to a device capable of providing an interface between one or more processor pins and an external device capable of inputting and/or outputting binary data.

In an example embodiment, one or more applications (e.g., Applications 270a and 270b) may be deployed in the CCE 50. The applications 270 may be device applications associated with a medical device, such as a MCT device that is configured to send and receive data to and from the key service 110. In an example embodiment, an application 270 and/or the MCT device may include a backend component (e.g., ECGB 150). For example, the proxy 180 may be included as a backend component as a library. In an example, the ECGB 150 allows communication requests to come in directly to the backend.

The CCE 50 provides on demand availability of computer system resources like processing and storage as well as computing services, like software, analysis, and analytics. In the examples illustrated herein, server(s) may be cloud servers and database(s) may be cloud databases that are deployed, delivered, and accessed in cloud. For example, cloud server(s) may be virtual (e.g., non-physical) servers running in a cloud computing environment. Cloud server(s) operate like physical servers and perform similar functions, such as storing data and running applications. Cloud database(s) may organize, and store structured, unstructured, and semi-structured data like a traditional on-premises database.

Referring now to FIG. 3, the key service 110 may provide services after mutual authentication. For example, the key service may be a cloud-based service where data security and privacy are critical. In an example, the key service 110 may adopt mutual authentication to secure communications between the key service 110 and a manufacturer's external system (e.g., EMS 130). As described herein, the key service 110 may provide keys for MCT devices, which are made and/or manufactured by one or more manufacturers that have their own systems. The portion of the EMS 130 that communicates with the key service 110 is typically executed, hosted and/or run outside of a care provider network, such as a hospital or treatment center.

Mutual authentication allows the key service 110 and the EMS 130 to communicate securely (e.g., secure data communication) via a network, such as the internet. As illustrated in FIG. 3, on the server side, the key service may install a domain name password to secure a domain name. On the client side, a manufacturing system (e.g., EMS 130) may install a client token to identify the client. In an example, each manufacturer or manufacturing system (e.g., EMS 130) may have its own client token. After mutual authentication is completed, the communication channel between the server side and the client side is secured. Typically, both the domain name password and the client token need to be regularly renewed. For example, the domain name password and the client token may require a renewal after a predetermined amount of time, activity or a combination thereof.

FIG. 3 illustrates a flowchart of an example mutual authentication process, which authenticates both the client and server. For example, an EMS 130 may start the mutual authentication process by sending or starting a handshake request 302 to the key service 110, as illustrated at arrow 310. The handshake request 310 may be communicated using Hypertext Transfer Protocol (“HTTP”), which may be congruent with the language used by web browsers to send and retrieve data. After receiving the handshake request, the key service 110 may present a server password 304 to the EMS at arrow 320.

The server password 304 may be validated with a password-token authority 360 at arrow 325. The EMS 130 may present a client token 306 to the key service 110 at arrow 330. In an example, the EMS 130 may present the client token 306 after the server password is validated at arrow 325. Then, the key service 110 may validate the client token 306 with the password-token authority 360 at arrow 335. If both the server password 304 and the client token 306 are successfully validated, the key service 110 may send requests/responses 308 to the EMS 130 at arrow 340.

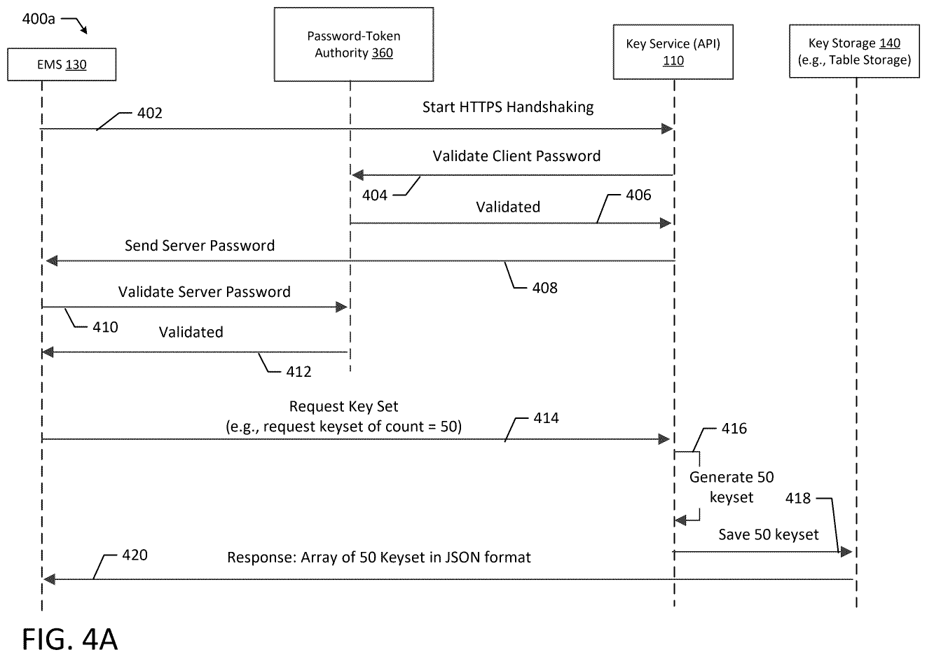

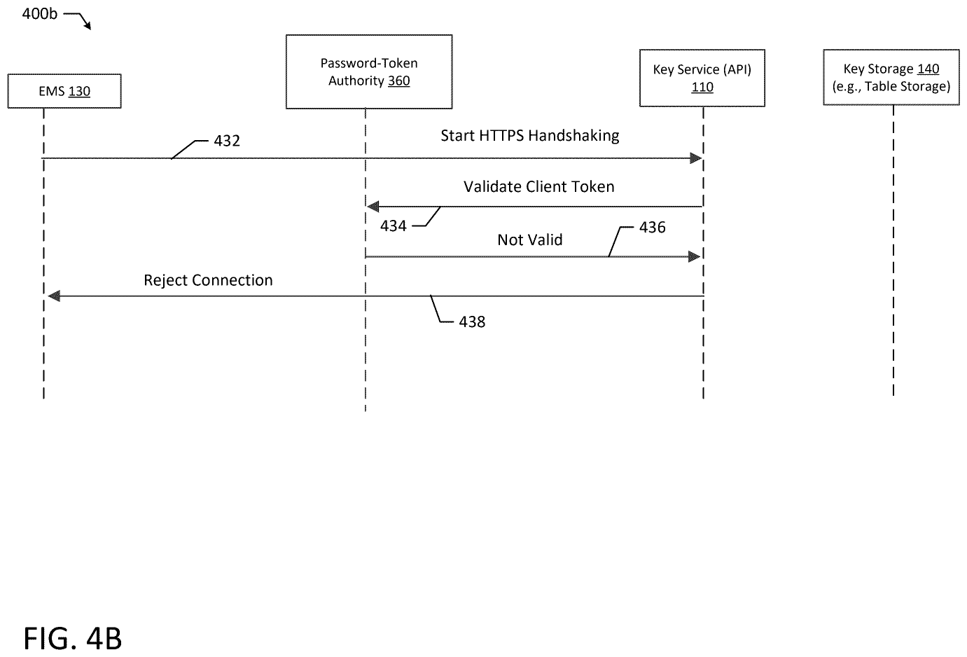

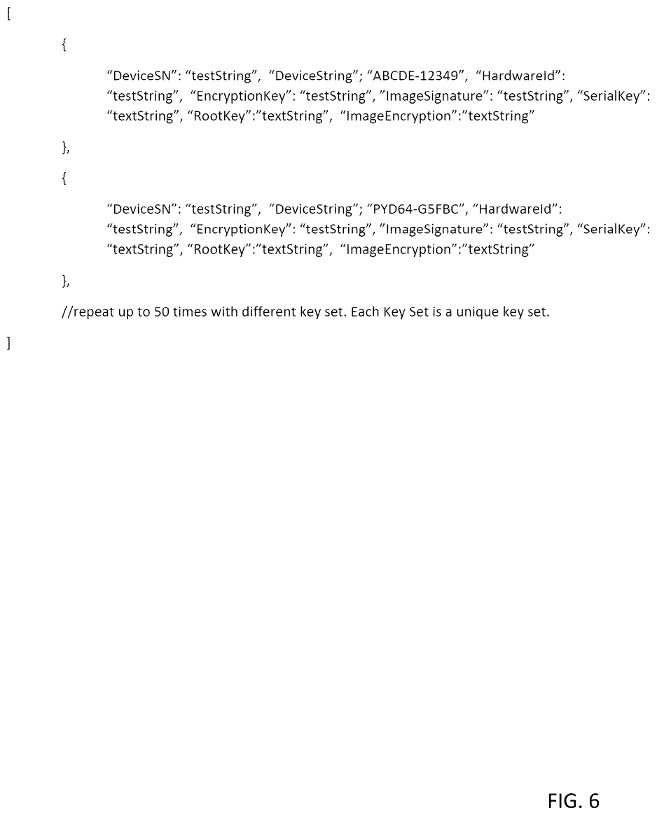

Obtaining the keyset 105 by the MCT 130 may include the use of an application program interface (“API”). In an example, the key service 110 may provide an API endpoint that provides generated keysets. The API endpoint may accept an integer count and may return that quantity of freshly generated keysets 105 (e.g., as long as the quantity of keysets 105 is within prescribed limits). The API may be defined for the EMS 130 to obtain one or more keysets 105. In an example, API may be a representational state transfer (REST) based application program interface (API), such as a RESTful API. Specifically, the key service 110 may provide a RESTful API that allows an EMS 130 to specify the quantity of keysets 105 to be generated. In an example, the value of the count may range from zero to one thousand (e.g., 0 to 1,000). It should be appreciated that other maximus, ranges or thresholds may be applied to limit or restrict the quantity of keysets 105 that can be generated per request. In an example, the return from the API call may be an array of keysets 105 may allow the token store 185 to communicate using Hypertext Transfer Protocol (HTTP), which may be congruent with the language used by web browsers to send and retrieve data. Jumping forward briefly to FIG. 6, an example keyset array is illustrated with respect to the example of FIG. 4A described in more detail below. As illustrated in FIG. 6, various keys such as “Device SN”, “HardwareID”, “EncryptionKey”, etc. are provided in the keyset array.

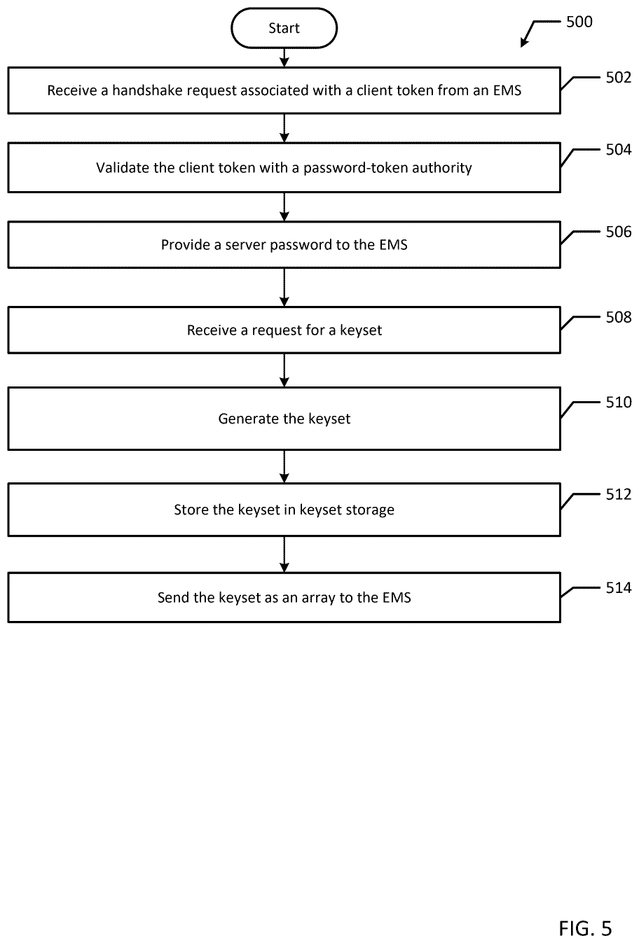

FIG. 4A is a flow diagram of an example process 400a for a successful mutual authentication between an external manufacturing system and a key service using a password-token authority and table storage. Conversely, FIG. 4B is a flow diagram of a process 400b for a failed mutual authentication. FIG. 5 illustrates a flowchart of an example method 500 for keyset generation and management. FIGS. 4A, 4B and 5 are described together below.

Jumping ahead to FIG. 5, which illustrates a flowchart of an example method 500 for keyset generation and management, will be described with additional references made to FIGS. 3, 4A and 4B. Although the example method 500 is described with reference to the flowchart illustrated in FIG. 5, it will be appreciated that many other methods of performing the acts associated with the method 500 may be used. For example, the order of some of the blocks may be changed, certain blocks may be combined with other blocks, and some of the blocks described are optional. The method 500 may be performed by processing logic that may comprise hardware (circuitry, dedicated logic, etc.), software, or a combination of both.

In the illustrated example, the method includes receiving a handshake request associated with a client token from an EMS (block 502). For example, a key service 110 may receive a handshake or handshaking request 302 from an EMS 130. The handshaking request 302 may be sent using Hypertext Transfer Protocol (HTTP), which may be congruent with the language used by web browsers to send and retrieve data. In other examples, other communication protocols may be used to send the handshaking request 302. Similarly, as illustrated in FIG. 4A, the EMS 130 initiates the HTTPS handshaking request 302 at arrow 402.

Then, method 500 includes validating the client token with a password-token authority (block 504). For example, the key service 110 may receive a client token 306 from the EMS 130 and then may validate the client token 306 with a password-token authority 360. Similarly, as illustrated in FIG. 4A, the EMS 130 may send the client token 306 along with the handshaking request 302 at arrow 402 where it is validated with the password-token authority 360 at arrow 404.

Method 500 also includes providing a server password to the EMS (block 506). For example, the key service 110 may present the server password 304 to the EMS 130. A server password 304 may be a digital file that verifies the identity of a web server and allows for encrypted communication between a server and a client (e.g., EMS 130). Server passwords 304 are essential for securing online communications, protecting data privacy, and ensuring the integrity of data. Similarly, as illustrated in FIG. 4A, once the client token 306 is validated at arrow 406, the key service 110 may send the server password 304 to the EMS 130 at arrow 408 where it is validated with the password-token authority 360 at arrow 410.

Then, method 500 includes receiving a request for a keyset (block 508). For example, key service 110 may receive a request 308a for a keyset 105. The request 308a may include a request count that indicates the quantity of keysets to be generated. For example, referring to the specific example in FIG. 4A, once the server password 304 is validated at arrow 412, the EMS 130 may request fifty keysets 105 (e.g., keyset request count=50) at arrow 414.

Next, method 500 includes generating the keyset (block 510). For example, the key service 110 may generate the keyset 105. Each keyset may include a plurality of keys that include one or more of a device identifier, a hardware identifier, an encryption key, an image authentication key, an image signature, a serial key, a root key, and an image encryption key. Specifically, each keyset 105 may be associated with a specific medical device, such as an MCT device. For example, the keysets may be unique for each unique device identifier (e.g., device serial number). In some instances, a medical device may be pre-loaded with a patient-specific keyset 105, such that the device has been ordered for and will be used on a specific patient. In the specific example illustrated in FIGS. 4A, the key service 110 generates fifty different keysets 105.

The device identifier may be a unique device serial number. For example, the device identifier may be represented by a 6-byte number in binary ECG data or may be displayed in human readable format. For example, a device serial number may have the human readable format: XXXX-XXXY where (i) each “X” is a character from the set “0123456789ABCDEFGHIJKMNPRSTUVWXYZ” which are alphanumeric characters that disclose “ILO” and (ii) each “Y” is a character from “89ABCDEF.” In an example, the key service 110 may return both binary and human-readable formats for the device identifier or serial number. In an example, the device identifier is provided by the random number generator 125 or a counter source.

The hardware identifier may be a unique ID programmed into the medical device, such as an MCT device or recorder. The hardware identifier may be used to track product and component lifecycle information, especially during refurbishment, updates, recalls or the like. The hardware identifier, such as a device serial number may be a ten-digit alpha numeric number. In an example, the device identifier is provided by the random number generator 125 or a counter source. Similarly, a medical device, such as a recorder, may have a recorder ID that follows the recorder through the recorder's lifecycle including refurbishments.

The encryption key may be a private symmetric key to encrypt data transmitted from a medical device to the ECGBE 150. The encryption key may be provided by a random source, such as from a cryptographically secure random number generator. Randomly generated keys, such as the encryption key, may be generated using a random number generator (“RNG”), a pseudorandom number generator (“PRNG”), or a combination thereof.

The image authentication key may be an image signature, such as an elliptic curve digital signature algorithm (“ECDSA”), which is a digital signature algorithm (“DSA”) which uses keys derived from elliptic curve cryptography (“ECC”). The image authentication key or image signature may be used to determine the authenticity of the medical device firmware image (e.g., recorder firmware image). The image authentication key and/or image signature may be provided by a configured source.

The serial key may be a symmetric communication key, such a universal asynchronous receiver/transmitter (“UART”) symmetric communication key. UART defines a protocol, or set of rules, for exchanging serial data between devices. In an example, the serial key is for use by a manufacturing station and command line interface communication. The serial key may be the same for all of an EMS's devices and may be used at or by recorder test stations. The serial key may be provided by a configured source.

The root key or root of trust key may be a Rivest-Shamir-Adleman (“RSA”) key that is used to establish root of trust. The root key may be created utilizing RSA encryption or another type of asymmetric encryption. The root key may also be used to sign a bootloader. In an example, an RSA key may be created and published as a public key based on two large prime numbers, along with an auxiliary value. The prime numbers are kept secret and messages, and information can be encrypted by anyone, via the public key, but can only be decrypted by a party that has the private key. The root key may be the same key used across all devices (e.g., may be the same for all of an EMS's devices). The root key may be provided by a configured source.

The image encryption key may be an AES-GCM 128 key where GCM refers to a Galois/Counter mode of operation for key cryptography. For example, GCM may use a block cipher with a block size of 128 bits (commonly AES-128) operated in counter mode for encryption. The image encryption key may encrypt the image stored in external flash memory of the medical device, such as an MCT device or recorder. The image encryption key may be provided by a configured source.

After generating the keyset, method 500 includes storing the keyset in keyset storage (block 512). For example, the key service 110 may store the keyset(s) 105 in key storage 140. The key storage 140 may be persistent storage and may use table storage. The key storage 140 may be persistent storage to permanently store data. Table storage may store non-relational structured data in the cloud computing environment 50. Because table storage is schemeless, table storage may be easy to adapt and access to table storage is fast and cost-effective for many types of applications. Typically, table storage provides storage at a lower in cost than traditional SQL for similar volumes of data. As illustrated in FIG. 4A, the key service 110 generates fifty keysets 105 at arrow 416.

Then, method 500 includes sending the keyset as an array to the EMS (block 514). For example, the key service 110 may send the keyset 105 as an array to the EMS 130. The array may be formatted as a JSON array. It should be appreciated that other formats may be used to save, store or provide the keysets 105.

As illustrated in FIG. 4A, the keyset 105 may be sent as a response 308b directly from the key storage 140 and/or table storage at arrow 420. In the illustrated example, the fifty keysets 105 are provided to the EMS 130 where they can be applied to each specific device, such as an MCT device.

As illustrated in FIG. 4B, in some examples the client token 306 is either invalid or cannot be validated/authenticated. For example, referring back to FIG. 5, the flow chart of FIG. 4B illustrates blocks 502 and 504 of method 500, but the validation at block 504 fails. Specifically, similar to FIG. 4A, the EMS 130 initiates a handshaking request 302 at arrow 432. Then, the EMS may send the client token 306 along with the handshaking request 302 at arrow 432 where it is reviewed for validation with the password-token authority 360 at arrow 434. In the example illustrated in FIG. 4B, the client token 306 is determined to be “not valid” at arrow 436 by the password-token authority 360. After determining that the client token 306 was invalid and could not be authenticated, the key service 110 rejects the connection with the EMS 130 at arrow 438.

In the examples illustrated in FIG. 3, FIGS. 4A-4B, and FIG. 5, if either the client token 306 or the server password 304 fails, the communication between the key service 110 and EMS 130 is ended thereby protecting any private or patient data from being conveyed to an unauthorized party. In each of the illustrated examples, an EMS 130 may perform an API request to the key service 110 to obtain a plurality of keysets. The key service 110 may obtain a master key for generating keys (e.g., from Azure Key Value) and then may obtain the next value in the random number generator 125 (e.g., from Azure Storage). The value may be incremented once obtaining the master key. In an example, the key service 110 may generate the keysets and respond to the EMS 130 with the requested keysets, which are also sent to a key storage database. In some examples, upwards of a thousand keysets may be generated at a time and may be programed into medical devices. When the EMS 130 is down to a minimum amount of remaining keysets or the amount of remaining keysets hits a lower predetermined threshold (e.g., 200 remaining keysets), the EMS 130 may request another batch of keysets 105.

Keysets 105 may be provisioned for the medical devices. For example, a singed bootloader may be loaded into an MCU, then the root of trust or root key may be setup and the keyset 105 may be installed. Then, the provisioning includes transferring the MCU to a secure state and loading an application image into external flash memory. Once the bootloader starts, the bootloader verifies the authenticity of the image stored in the external flash memory using the image authentication key and ECDSA. The image is then decrypted and stored in the MCU using the root of trust key. Finally, the application image is run.

When setting up a medical device or kit at a health care provider (“HCP”), a device serial number may be entered into patient registration. The HCP may be provided feedback that the serial number (e.g., device identifier) is valid and possibly whether the device is still within manufacturing warranty. Then, once the medical device is connected to the network (e.g., Internet) through an associated mobile device, the ECGB 150 connects with the key storage to get the keyset 105 allowing the ECGB to authenticate and decrypt the ECG data. In an example, the ECG data may be cached to avoid overloading any of the associated servers. Communication between the

While the invention has been particularly shown and described as referenced to the embodiments thereof, those skilled in the art will understand that the foregoing and other changes in form and detail may be made therein without departing from the spirit and scope.

What is claimed is:

1. A system comprising:
a key service, that is configured to:
authenticate requests from an external manufacturing system (“EMS”);
receive a request for generating a keyset;
generate the keyset;
send the keyset to the EMS;
send the keyset to a persistent cloud storage system,
wherein an external cloud system accesses the keyset for use in secure communication with a hardware device.

2. The system of claim 1, wherein the keyset includes a plurality of keys.

3. The system of claim 2, wherein the plurality of keys includes at least seven keys.

4. The system of claim 1, further comprising a key storage memory configured to store the keyset.

5. The system of claim 4, wherein the key storage memory is configured to store each key of the keyset together as a unit in key storage memory.

6. The system of claim 4, wherein the key storage memory is persistent memory.

7. The system of claim 1, wherein the keyset includes at least one of a device identifier, a hardware identifier, an encryption key, an image authentication key, an image signature, a serial key, a root key, and an image encryption key.

8. The system of claim 1, wherein the key service is configured to send the generated keyset as an array, wherein the array is a JSON formatted array.

9. A method comprising:
authenticating, by a key service, requests from an external manufacturing system (“EMS”);
receiving, by the key service, a request for generating a keyset;
generating, by the key service, the keyset;
sending the keyset to the EMS;
sending the keyset to a persistent cloud storage system,
wherein an external cloud system accesses the keyset for use in secure communication with a hardware device.

10. The method of claim 9, wherein the keyset includes a plurality of keys.

11. The method of claim 10, wherein the plurality of keys includes at one of a device identifier, a hardware identifier, an encryption key, an image authentication key, an image signature, a serial key, a root key, and an image encryption key.

12. The method of claim 9, wherein each key of the keyset is stored together as a unit in key storage memory.

13. The method of claim 12, wherein the key storage memory is persistent memory.

14. The method of claim 9, wherein the array is a JSON formatted array.

15. The method of claim 9, wherein a respective key of the keyset has a source, and wherein the source is one of a configured source and a random source.

16. The method of claim 15, wherein the configured source is configured to provide preloaded keys and manually set keys, wherein the random source includes a cryptographically secure random number generator.

17. A non-transitory machine-readable medium storing code, which when executed by a processor causes a key service to:
authenticate requests from an external manufacturing system (“EMS”);
receive a request for generating a keyset;
generate the keyset;
send the keyset to the EMS;
send the keyset to a persistent cloud storage system,
wherein an external cloud system accesses the keyset for use in secure communication with a hardware device.

18. The non-transitory machine-readable medium of claim 17, wherein the keyset includes a plurality of keys.

19. The non-transitory machine-readable medium of claim 18, wherein the plurality of keys includes at one of a device identifier, a hardware identifier, an encryption key, an image authentication key, an image signature, a serial key, a root key, and an image encryption key.

20. The non-transitory machine-readable medium claim 18, wherein each key of the keyset is stored together as a unit in key storage memory, and wherein the key storage memory is persistent memory.
